(19) United States
(12) Patent Application Publication
(10) Pub. No.: US 2004/0195084 A1
(43) Pub. Date: Oct. 7, 2004

(54) Process for separating sec-butanol from ethyl acetate

(76) Inventors: Stephen Warren Hetherington, Hampshire (GB); Witold Franciszek Pacynko, East Yorkshire (GB)

Correspondence Address: NIXON & VANDERHYE, PC, 1100 N GLEBE ROAD, 8TH FLOOR, ARLINGTON, VA, 22201-4714, US

(21) Appl. No.: 10/485,367

(22) PCT Filed: Jul. 25, 2002
(86) PCT No.: PCT/GB02/03411

(30) Foreign Application Priority Data
Jul. 30, 2001 (GB) 0118553.7

(51) Int. Cl. B01D 3/42; C07C 27/26

(52) U.S. Cl. 203/2; 203/99; 203/DIG. 19; 203/DIG. 21; 568/913

(57) ABSTRACT

A process for separating secondary-butanol impurity from ethyl acetate (EtAc) by feeding the impure EtAc to a distillation column operating at a pressure of less than 1 bar absolute to provide (1) a stream comprising EtAc as a major component and (2) a residue or a second stream comprising at least some sec-butanol from said impure EtAc. The process can be applied to purifying EtAc derived from (a) catalytic reaction of ethylene with acetic acid followed by (b) a hydrogenation step. The 2-butanone impurity produced in step (a) is difficult to separate from EtAc, and step (b) converts it to sec-butanol which can be separated by the reduced pressure fractionation of the invention.

This invention relates to process for removing a sec-butanol impurity from a product stream comprising ethyl acetate.

Ethyl acetate may be produced by several methods known in the art. One such method comprises reacting ethylene with acetic acid in the presence of an acidic catalyst, for example, an acidic heteropolyacid catalyst. In a second such method, ethyl acetate is produced by converting an alcohol feedstock by i) dehydrogenation, ii) oxidation, iii) reaction with an aldehyde or iv) oxidation to the corresponding aldehyde followed by the Tischenko reaction (see, for example, EP 0992484).

These reactions can produce a product stream comprising ethyl acetate, unreacted starting materials, a number of aldehyde and ketone impuries, such as acetaldehyde, methyl i-propyl ketone, butyraldehyde, methyl propyl ketone, methyl i-butyl ketone, methyl-s-butyl ketone, methyl i-pentyl ketone, methyl ethyl ketone (MEK), as well as variety of C8, branched and higher alkenes, such as methyl heptene and dimethyl hexene. The unreacted starting materials are recovered from the product stream, and recycled to the reactor. The ethyl acetate may be recovered from the remainder of the product stream, for example, by distillation. Unfortunately, some aldehyde and/or ketone impurities, such as MEK, have a boiling point that is very similar to the boiling point of ethyl acetate and, for example, it is difficult to reduce or maintain the MEK concentrations of the final product to below 50 ppm using this method.

Various attempts have been made to reduce the concentration of such aldehyde and/or ketone impurities in alkyl alkanoate streams further. As aldehydes and ketones may form azeotropes with alkyl alkanoates, attempts have been made to separate the impurities using azeotropic distillation (see for example EP 0151886).

EP 0992484 describes a process in which aldehyde and/or ketone impurities are removed from an alkyl alkanoate product stream by contacting the impure alkyl alkanoate product stream with a selective hydrogenation catalyst of, for example, ruthenium in the presence of hydrogen. The hydrogenation reaction is preferably carried out at elevated pressures of 25 to 50 barg. Under the reaction conditions, the aldehyde and/or ketone impurities are selectively hydrogenated to the corresponding alcohols, leaving the alkyl alkanoate substantially unreacted. As many alcohols tend to boil at a very different temperature to alkyl alkanoates, the former can be separated by simple distillation.

However, it has been found that in the case of ethyl acetate and MEK, the alcohol formed by hydrogenation of MEK, sec-butanol, is harder to separate from the desired ethyl acetate, because the ethyl acetate and sec-butanol form a pinch under conventional distillation conditions at atmospheric pressure. A column is said to be pinched when the component balance line is too close to the equilibrium curve. The practical significance is that very little separation is taking place and the use of many separation stages may only result in a very small change in composition. Hence it becomes very difficult to reduce the sec-butanol in the ethyl acetate to a low level. A pinch can be remedied by increasing the reflux and reboil, thus drawing the component balance line and the equilibrium curve further apart, but this is achieved at a penalty of significantly greater energy consumption.

We have now found that the pinch can also be eased, and the sec-butanol level in ethyl acetate can be reduced further, i.e.. ethyl acetate of higher purity can be obtained, by the use of reduced pressure in the corresponding distillation column.

Accordingly, the present invention provides a process for separating ethyl acetate from sec-butanol, said process comprising:

taking a product stream comprising ethyl acetate and sec-butanol,

feeding the product stream to a distillation column,

operating the distillation column at a pressure of less than 1 bar absolute to give an ethyl acetate stream and a sec-butanol stream.

Thus one aspect of the present invention provides a process for separating sec butanol impurity from ethyl acetate, said process comprising:

feeding to a distillation column a product stream comprising at least ethyl acetate and sec-butanol,

operating the distillation column at a pressure of less than 1 bar absolute to provide at least

(1) a stream comprising ethyl acetate as a major component and

(2) a residue or a second stream which residue or second stream comprises at least some sec-butanol from said product stream

Under the reduced pressure conditions of the present invention it has been found that the ethyl acetate/sec-butanol pinch can be eased and improved separation can be obtained. Ethyl acetate product is thus removed as an overhead stream.

Preferably the distillation column of the present invention is operated so that the pressure in the column lies in the range 0.01 and 0.95 bar absolute, more preferably between 0.1 and 0.7 bar absolute, and most preferably between 0.3 and 0.5 bar absolute.

The feed to the distillation column will preferably be introduced between one-quarter and three-quarters of the way up the column, more preferably in the central third. Most preferably the feed will be between one-third and halfway up the column from the base. It will be readily apparent to one skilled in the art that the exact operating conditions of the column may depend on a number of factors, for example, the number of stages, the purity of the feed and the purity of product desired. For example, in a distillation column with 25 theoretical stages, the feed to the column is preferably located 15 stages below the top of the column. Preferably the column is operated at a reflux ratio of 2:1. Under these conditions and at a pressure of 0.5 bara, then the temperature at the head of the column, containing largely EtAc, would be 57.3 deg C.

The level of sec-butanol in the final EtAc product may be further reduced by control of the base purge of the distillation column. In general an increased purge rate will lead to a reduction in the base level of sec-butanol and hence a reduced level in the heads, but at the expense of loss of other products in the purge.

It has now been found that under the conditions of operation of the distillation column that there is a second effect of varying the purge rate which favours reduced purge rates. It has been found that sec-butanol may react in the base of the column with either acetic acid (which is produced in the column by hydrolysis of the EtAc) or directly with EtAc. Both of these reactions lead to production of sec-butylacetate, with by-products of water and ethanol respectively. The sec-butylacetate formed may be easily separated from the EtAc by the column of the current invention. With reduced purge rates any sec-butanol in the base will have an increased residence time and hence the extent of reaction will be increased.

Hence there are two competing processes determining the level of sec-butanol in the base of the column. Reduction of the base level of sec-butanol can be achieved directly by increased purge rate. However reduction of the base level of sec-butanol can also be achieved by increased reaction of the sec-butanol. Due to the competing process of sec-butanol reaction, the purge rate required in the distillation may be expected to be less than that required in the absence of the competing process.

Preferably, purge rates are adjusted throughout the life of a catalyst. For example, initial purge rates may be low as a fresh catalyst may produce less MEK, but as the catalyst ages it may produce increased MEK (and hence sec-butanol) and purge rates maybe increased.

It will also be apparent that the rate of reaction of sec-butanol with acetic acid will depend on the amount of acetic acid in the base of the column. It is therefore possible to control the rate of reaction of the sec-butanol with acetic acid by changing the amount of acetic acid in the base of the column. For example, increased reaction rates can be obtained by addition of further acetic acid directly in the base of the column or to the EtAc/sec-butanol stream prior to the column. This will also affect the purge rate required in the distillation column.

It will be readily apparent to one skilled in the art that the process of the present invention using distillation under reduced pressure is readily applicable to separation of an ethyl acetate/sec-butanol stream derived from any source.

In a preferred embodiment of the present invention the ethyl acetate/sec-butanol stream is derived from a stream comprising ethyl acetate and methyl ethyl ketone (MEK), wherein the MEK has been hydrogenated to produce the sec-butanol.

In a further preferred embodiment the ethyl acetate/MEK stream has itself been derived from the reaction of ethylene and acetic acid, or from the conversion of an alcohol feedstock to ethyl acetate by i) dehydrogenation, ii) oxidation, iii) reaction with an aldehyde or iv) oxidation to the corresponding aldehyde followed by the Tischenko reaction.

For example, the reaction conditions necessary for producing ethyl acetate from the reaction between ethylene and acetic acid are well-known in the art, and are described by way of example in GB-A-1259390. As well as ethyl acetate, the product stream comprises aldehyde and/or ketone impurities. Examples of aldehyde impurities include acetaldehyde, propionaldehyde, butyraldehyde, valeraldehyde. Examples of ketone impurities include methyl iso-propyl ketone, methyl propyl ketone, methyl iso-butyl ketone, methyl-sec-butyl ketone, methyl-iso-pentyl ketone and MEK (methyl ethyl ketone). These impurities may form more than 5 ppm, preferably, 5 to 1000 ppm, more preferably, 5 to 500 ppm of the product stream, prior to treatment.

The MEK in this product stream is hydrogenated to produce sec-butanol, for example, by contacting all or a part of the product stream comprising MEK with hydrogen in the presence of a selective hydrogenation catalyst. Other impurities that comprise the product stream may also be selectively hydrogenated. The selective hydrogenation catalyst is selected to be relatively active with respect to the hydrogenation of aldehyde and/or ketone carbonyl groups, but relatively inactive with respect to the hydrogenation of alkyl alkanoate carbonyl groups. Suitable catalysts comprise transition metals such as nickel, palladium, platinum, ruthenium, rhodium and rhenium. Such catalysts may be supported, for example, on alumina, silica or carbon. The metal loadings on such supported catalysts may range from 0.1 to 50 wt %, preferably, 0.5 to 10 wt %. Examples of specific catalysts include Ni on alumina or silica, Ru on carbon or silica, Pd on carbon, Rh on carbon and Pt on carbon. In a preferred embodiment, a 3-5wt % Ru catalyst supported on carbon or silica is employed.

The selective hydrogenation step may be carried out in the presence of any suitable solvent, for example, water, and/or alkyl alkanoate.

The hydrogen employed in the selective hydrogenation step may be employed in pure or impure form. Optionally, an inert gas such as nitrogen may be co-fed to the reaction.

The selective hydrogenation step may be carried out at 40 to 120° C., preferably, 80-100° C. The combined partial pressure of the product stream and hydrogen employed in the hydrogenation step may range from 1 to 80 barg (bar gauge), preferably, 1 to 50 barg, more preferably, 1 to 40 barg.

The mole ratio of the product stream to hydrogen employed may be 1000:1 to 5:1, preferably from 100: 1 to 10:1, for example, 60:1.

The product stream may be passed over the selective hydrogenation catalyst at a liquid hourly space velocity (LHSV) of 0.1 hr⁻¹ to 20 hr⁻¹, preferably, 1 hr⁻¹ to 15 hr⁻¹, and most preferably 5 to 10 hr⁻¹.

Under the selective hydrogenation conditions used in the preferred process of the present invention, the MEK impurity is selectively hydrogenated to sec-butanol. Any other aldehyde and/or ketone impurities are also hydrogenated to their corresponding alcohols. The hydrogenated stream so produced comprises ethyl acetate and sec-butanol. In certain embodiments this stream may be further treated prior to feeding to the distillation column operating at a pressure of less than 1 bar absolute as described previously. Preferably the stream may be treated to remove any unreacted hydrogen. Hydrogen separation may be achieved, for example, by using a flash tank or a separation column. The separated hydrogen may be purged or recycled for re-use. In another embodiment the stream may undergo further separation stages prior to the separation of the ethyl acetate and sec-butanol, to remove other components, such as, for example, water, ethanol and other alcohols formed in the hydrogenation reaction. Aqueous phases may be removed, for example, using a settling unit. Most preferably the hydrogenated stream may be mixed with water and then fed to a decanter. The aqueous phase is allowed to separate, thus removing a proportion of the ethanol. The oil rich phase comprising a major proportion of ethyl acetate may also be fed to a distillation column for further separations prior to the stream comprising ethyl acetate and sec-butanol being fed to the distillation column at reduced pressure of the present invention.

### EXAMPLE 1

Experiments were run on a 50-tray pilot plant distillation column to test the efficiency for removing s-BuOH froth the final product. Small samples could be taken at intervals from both the reboiler sump at the base of the column and the reflux drum at the top, and from above various intermediate trays within the distillation column. The column reboiler was charged with the mixture in table 1 below:

TABLE 1

| | |
|---|---|
| Component | m/m % |

| | |
|---|---|
| Ethyl acetate | 62 |
| Ethyl propionate | 10 |
| s-Butyl acetate | 3 |
| s-BuOH | 9 |
| Misc. hydrocarbons | balance |

S-BuOH was dosed into a mixer such that the feed to the column typically contained 330 ppm s-BuOH. The calculated feed rate averaged some 4050 g/hr, entering via the center feed point (i.e. tray 20) and the column was operated at a reflux ratio of 2:1, with the column pressure being measured at the base. At a pressure of 1.06 bara an average s-BuOH distillate concentration of approximately 20 ppm was achieved. When the pressure was reduced to 0.52 bara the concentration of s-BuOH in the distillate reduced to an average of 7 ppm

The experiment was then run with a reduced feed supply rate of 3230 g/hr such that the base contained a reduced concentration of s-BuOH (<5m/m%). The column was operated at between to 0.54 and 0.6 bara and under these conditions the distillate no longer contained any detectable s-BuOH (i.e. 1 ppm or less).

### EXAMPLE 2

### s-Butanol Esterification and Trans-esterification in the Base of “D7600”

D7600 is a commercial 50 tray distillation column used for the purification of ethyl acetate. A small amount of acetic acid is produced via ethyl acetate hydrolysis and accumulates in the base of the distillation column. It was calculated that, assuming a 17 CuM hydrogenation reactor for converting the MEK to sec-butanol, a measured flow of 21 kgs/hr of acetic acid reaches a steady state concentration of approximately 18 wt % assuming a base purge of 130 kgs/hr. The residence times observed in the base of the column are also dictated by the purge rate and lead to residence times of the order of 60 to 70 hours.

In order to further understand the rates of these processes a test was carried out on the pilot plant where the base composition of the distillation column was adjusted to increase both acid and s-BuOH in the base to 13.34% and 8.85%, respectively.

Table 2 below shows the composition change during the test:

TABLE 2

| | | | Corrected | Wt % | |
|---|---|---|---|---|---|
| | Starting | Wt % after | wt % after | change over | Molar |
| Component | wt % | 70 hours | 70 hours | 70 hours | change |
| Acetic Acid | 13.34 | 12.69 | 10.94 | −2.38 | −0.040 |
| s-BuAc | 3.12 | 10.56 | 9.12 | +6.00 | +0.052 |
| s-Butanol | 8.85 | 5.41 | 4.67 | −4.18 | −0.056 |
| Et Prop | 7.61 | 8.81 | 7.61 | — | — |

Ethyl propionate was expected not to be significantly effected by the reactions taking place and were therefore used as a means for adjusting the compositions for changing sump level.

The results show that the molar increase in s-BuAc is consistent with the decrease in s-Butanol. The data shows that when steady state is reached, using a residence time of 70 hours, 47% of the butanol will have reacted; the remainder will have been purged from the system. In addition the data shows that the loss of acid due to esterification is the predominant reaction accounting for 74% of the butanol reaction. The trans-esterification reaction is slower accounting for 26% of the butanol conversion.

### EXAMPLE 3

Using the 50 tray pilot plant distillation column referred to in Example 1, a series of tests was carried out using a 2:1 reflux ratio throughout. The results are shown in Table 3 below. Ethyl acetate containing the indicated quantity of sec-butanol impurity was fed to the column at the indicated rate. The concentration of sec-butanol impurity in the ethyl acetate distillate can be seen to fall dramatically to 7 ppm when the column pressure is reduced from 1 bara down to 0.52 bara. Reduction of the feed rate results in a further drop in sec-butanol level in the distillate to less than 1 ppm.

TABLE 3

| | | Column | s-BuOH | | | |
|---|---|---|---|---|---|---|
| | Feed | Pressure | Feed | Base | Distillate | |
| Test No. | Rate | Bara | (ppm) | (%) | (ppm) | Precision |
| 1 | 3.8 | 1.0 | 30 | 2.3 | 5 | 2 |
| 2 | 3.8 | 1.0 | 320 | 4.5 | 15 | 3 |
| 3 | 3.8 | 1.0 | 320 | 2.5 | 10 | 2 |
| 4 | 4.0 | 1.0 | 330 | 8.0 | 20 | 2 |
| 5 | 4.0 | 0.52 | 330 | 9.0 | 7 | 2 |
| 6 | 4.2 | 0.54 | 330 | 5.0 | 0 | 2 |

1. A process for separating ethyl acetate from sec-butanol, said process comprising:
taking a product stream comprising ethyl acetate and sec-butanol, feeding the product stream to a distillation column, operating the distillation column at a pressure of less than 1 bar absolute to give an ethyl acetate stream and a sec-butanol stream.

2. A process for separating sec-butanol impurity from ethyl acetate, said process comprising:
feeding to a distillation column a product stream comprising at least ethyl acetate and sec-butanol, operating the distillation column at a pressure of less than 1 bar absolute to provide at least
(1) a stream comprising ethyl acetate as a major component and
(2) a residue or a second stream which residue or second stream comprises at least some sec-butanol from said product stream.

3. A process as claimed in claim 1 wherein the pressure in the distillation column lies in the range 0.01 and 0.95 bar absolute.

4. A process as claimed in claim 1 wherein the pressure in the distillation column lies in the range 0.1 and 0.7 bar absolute.

5. A process as claimed in claim 1 wherein the pressure in the distillation column lies in the range between 0.3 and 0.5 bar absolute.

6. A process as claimed in claim 1 wherein the product stream is fed to the distillation column between one-quarter and three-quarters of the way up the column.

7. A process as claimed in any claim 1 wherein the product stream is fed to the central third of the distillation column.

8. A process as claimed in any claim 1 wherein the ethyl acetate/sec-butanol stream is derived from a stream (3) comprising ethyl acetate and methyl ethyl ketone (MEK), wherein the MEK has been hydrogenated to produce the sec-butanol.

9. A process as claimed in claim 8 wherein the ethyl acetate I IVIEK stream (3) has itself been derived from the reaction of ethylene and acetic acid, or from the conversion of an alcohol feedstock to ethyl acetate by i) dehydrogenation, ii) oxidation, iii) reaction with an aldehyde or iv) oxidation to the corresponding aldehyde followed by the Tischenko reaction.

10. A process as claimed in claim 8 wherein the MEK in the product stream (3) is hydrogenated to produce sec-butanol by contacting all or a part of product stream (3) with hydrogen in the presence of a selective hydrogenation catalyst.

11. A process as claimed in claim 9 wherein the selective hydrogenation catalyst comprises a 3-Swt % Ru catalyst supported on carbon or silica.

12. A process for purifying ethyl acetate substantially as hereinbefore described in the Examples.

13. Ethyl acetate prepared by the process described in claim 1.
